(19) United States
(12) Patent Application Publication
(10) Pub. No.: US 2013/0237856 A1
(43) Pub. Date: Sep. 12, 2013

(54) Apparatus and Method for Mitigating Noise Affecting a Transcutaneous Signal

(71) Applicants: Gary P. Warren, (US); Matthew S. Alley, (US); IVWATCH, LLC, Williamsburg, VA (US)

(72) Inventors: Gary P. Warren, Williamsburg, VA (US); Matthew S. Alley, Sandston, VA (US)

(73) Assignee: IVWATCH, LLC, Williamsburg, VA (US)

(21) Appl. No.: 13/792,074

(22) Filed: Mar. 10, 2013

Related U.S. Application Data
(60) Provisional application No. 61/706,726, filed on Sep. 27, 2012.
(60) Provisional application No. 61/609,865, filed on Mar. 12, 2012.

(51) Int. Cl. A61B 5/00

(52) U.S. Cl. CPC A61B 5/443
USPC 600/476

(57) ABSTRACT

A system and method include a sensor overlying a target area of skin to aid in diagnosing subcutaneous fluid leakage. The sensor includes an absorbent that minimizes noise in detected electromagnetic radiation to make it easier to analyze a signal that is indicative of subcutaneous fluid leakage.

### CROSS-REFERENCES TO RELATED APPLICATIONS

This application claims the priority of U.S. Provisional Application No. 61/706,726, filed 27 Sep. 2012, and also claims the priority of U.S. Provisional Application No. 61/609,865, filed 12 Mar. 2012, each of which are hereby incorporated by reference in their entirety.

### STATEMENT REGARDING FEDERALLY SPONSORED RESEARCH OR DEVELOPMENT

Not Applicable

### BACKGROUND OF THE INVENTION

FIGS. 4 and 4A show a typical arrangement for intravascular infusion. As the terminology is used herein, “intravascular” preferably refers to being situated in, occurring in, or being administered by entry into a blood vessel, thus “intravascular infusion” preferably refers to introducing a fluid or infusate into a blood vessel. Intravascular infusion accordingly encompasses both intravenous infusion (administering a fluid into a vein) and intra-arterial infusion (administering a fluid into an artery).

A cannula 20 is typically used for administering fluid via a subcutaneous blood vessel V. Typically, cannula 20 is inserted through skin S at a cannulation or cannula insertion site N and punctures the blood vessel V, for example, the cephalic vein, basilica vein, median cubital vein, or any suitable vein for an intravenous infusion. Similarly, any suitable artery may be used for an intra-arterial infusion.

Cannula 20 typically is in fluid communication with a fluid source 22. Typically, cannula 20 includes an extracorporeal connector, e.g., a hub 20a, and a transcutaneous sleeve 20b. Fluid source 22 typically includes one or more sterile containers that hold the fluid(s) to be administered. Examples of typical sterile containers include plastic bags, glass bottles or plastic bottles.

An administration set 30 typically provides a sterile conduit for fluid to flow from fluid source 22 to cannula 20. Typically, administration set 30 includes tubing 32, a drip chamber 34, a flow control device 36, and a cannula connector 38. Tubing 32 is typically made of polypropylene, nylon, or another flexible, strong and inert material. Drip chamber 34 typically permits the fluid to flow one drop at a time for reducing air bubbles in the flow. Tubing 32 and drip chamber 34 are typically transparent or translucent to provide a visual indication of the flow. Typically, flow control device 36 is positioned upstream from drip chamber 34 for controlling fluid flow in tubing 34. Roller clamps and Dial-A-Flo®, manufactured by Hospira, Inc. (Lake Forest, Ill., USA), are examples of typical flow control devices. Typically, cannula connector 38 and hub 20a provide a leak-proof coupling through which the fluid may flow. Luer-Lok™, manufactured by Becton, Dickinson and Company (Franklin Lakes, N.J., USA), is an example of a typical leak-proof coupling.

Administration set 30 may also include at least one of a clamp 40, an injection port 42, a filter 44, or other devices. Typically, clamp 40 pinches tubing 32 to cut-off fluid flow. Injection port 42 typically provides an access port for administering medicine or another fluid via cannula 20. Filter 44 typically purifies and/or treats the fluid flowing through administration set 30. For example, filter 44 may strain contaminants from the fluid.

An infusion pump 50 may be coupled with administration set 30 for controlling the quantity or the rate of fluid flow to cannula 20. The Alaris® System manufactured by CareFusion Corporation (San Diego, Calif., USA) and Flo-Gard® Volumetric Infusion Pumps manufactured by Baxter International Inc. (Deerfield, Ill., USA) are examples of typical infusion pumps.

Intravenous infusion or therapy typically uses a fluid (e.g., infusate, whole blood, or blood product) to correct an electrolyte imbalance, to deliver a medication, or to elevate a fluid level. Typical infusates predominately consist of sterile water with electrolytes (e.g., sodium, potassium, or chloride), calories (e.g., dextrose or total parenteral nutrition), or medications (e.g., anti-infectives, anticonvulsants, antihyperuricemic agents, cardiovascular agents, central nervous system agents, chemotherapy drugs, coagulation modifiers, gastrointestinal agents, or respiratory agents). Examples of medications that are typically administered during intravenous therapy include acyclovir, allopurinol, amikacin, aminophylline, amiodarone, amphotericin B, ampicillin, carboplatin, cefazolin, cefotaxime, cefuroxime, ciprofloxacin, cisplatin, clindamycin, cyclophosphamide, diazepam, docetaxel, dopamine, doxorubicin, doxycycline, erythromycin, etoposide, fentanyl, fluorouracil, furosemide, ganciclovir, gemcitabine, gentamicin, heparin, imipenem, irinotecan, lorazepam, magnesium sulfate, meropenem, methotrexate, methylprednisolone, midazolam, morphine, nafcillin, ondansetron, paclitaxel, pentamidine, phenobarbital, phenytoin, piperacillin, promethazine, sodium bicarbonate, ticarcillin, tobramycin, topotecan, vancomycin, vinblastine and vincristine. Transfusions and other processes for donating and receiving whole blood or blood products (e.g., albumin and immunoglobulin) also typically use intravenous infusion.

Unintended infusing typically occurs when fluid from cannula 20 escapes from its intended vein/artery. Typically, unintended infusing causes an abnormal amount of the fluid to diffuse or accumulate in perivascular tissue and may occur, for example, when (i) cannula 20 causes a vein/artery to rupture; (ii) cannula 20 improperly punctures the vein/artery; (iii) cannula 20 backs out of the vein/artery; (iv) cannula 20 is improperly sized; (v) infusion pump 50 administers fluid at an excessive flow rate; or (vi) the infusate increases permeability of the vein/artery. As the terminology is used herein, “tissue” preferably refers to an association of cells, intercellular material and/or interstitial compartments, and “perivascular tissue” preferably refers to cells, intercellular material and/or interstitial compartments that are in the general vicinity of a blood vessel and may become unintentionally infused with fluid from cannula 20. Unintended infusing of a non-vesicant fluid is typically referred to as “infiltration,” whereas unintended infusing of a vesicant fluid is typically referred to as “extravasation.”

The symptoms of infiltration or extravasation typically include blanching or discoloration of the skin S, edema, pain, or numbness. The consequences of infiltration or extravasation typically include skin reactions such as blisters, nerve compression, compartment syndrome, or necrosis. Typical treatment for infiltration or extravasation includes applying warm or cold compresses, elevating an affected limb, administering hyaluronidase, phentolamine, sodium thiosulfate or dexrazoxane, fasciotomy, or amputation.

### BRIEF SUMMARY OF THE INVENTION

Embodiments according to the present invention include a sensor for evaluating an anatomical change over time in perivascular tissue. The sensor includes an emitter face, a detector face, and an absorber. The emitter face is configured to emit a first electromagnetic radiation signal. The detector face is configured to detect a second electromagnetic radiation signal. The second electromagnetic radiation signal is at least one of a reflection, scattering and redirection of the first electromagnetic radiation signal by the perivascular tissue. The absorber is configured to absorb a third electromagnetic radiation signal. The third electromagnetic radiation signal is at least one of a reflection, scattering and redirection of the first electromagnetic radiation signal by an epidermis overlying the perivascular tissue.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features, principles, and methods of the invention.

FIG. 1 is a schematic cross-section view illustrating an electromagnetic energy sensor.

FIG. 2 is a schematic cross-section view illustrating separation of the electromagnetic energy sensor shown in FIG. 1.

FIGS. 2A and 2B are schematic cross-section views illustrating alternative details of area II shown in FIG. 2.

FIG. 3 is a schematic cross-section view illustrating an embodiment of an electromagnetic energy sensor according to the present disclosure.

FIG. 3A is a plan view illustrating a superficies of the electromagnetic energy sensor shown in FIG. 3.

FIG. 4 is a schematic view illustrating a typical set-up for infusion administration.

FIG. 4A is a schematic view illustrating a subcutaneous detail of area IVA shown in FIG. 4.

In the figures, the thickness and configuration of components may be exaggerated for clarity. The same reference numerals in different figures represent the same component.

### DETAILED DESCRIPTION OF THE INVENTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment according to the disclosure. The appearances of the phrases “one embodiment” or “other embodiments” in various places in the specification are not necessarily all referring to the same embodiment, nor are separate or alternative embodiments mutually exclusive of other embodiments. Moreover, various features are described which may be exhibited by some embodiments and not by others. Similarly, various features are described which may be included in some embodiments but not other embodiments.

The terms used in this specification generally have their ordinary meanings in the art, within the context of the disclosure, and in the specific context where each term is used. Certain terms in this specification may be used to provide additional guidance regarding the description of the disclosure. It will be appreciated that a feature may be described more than one-way.

Alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term.

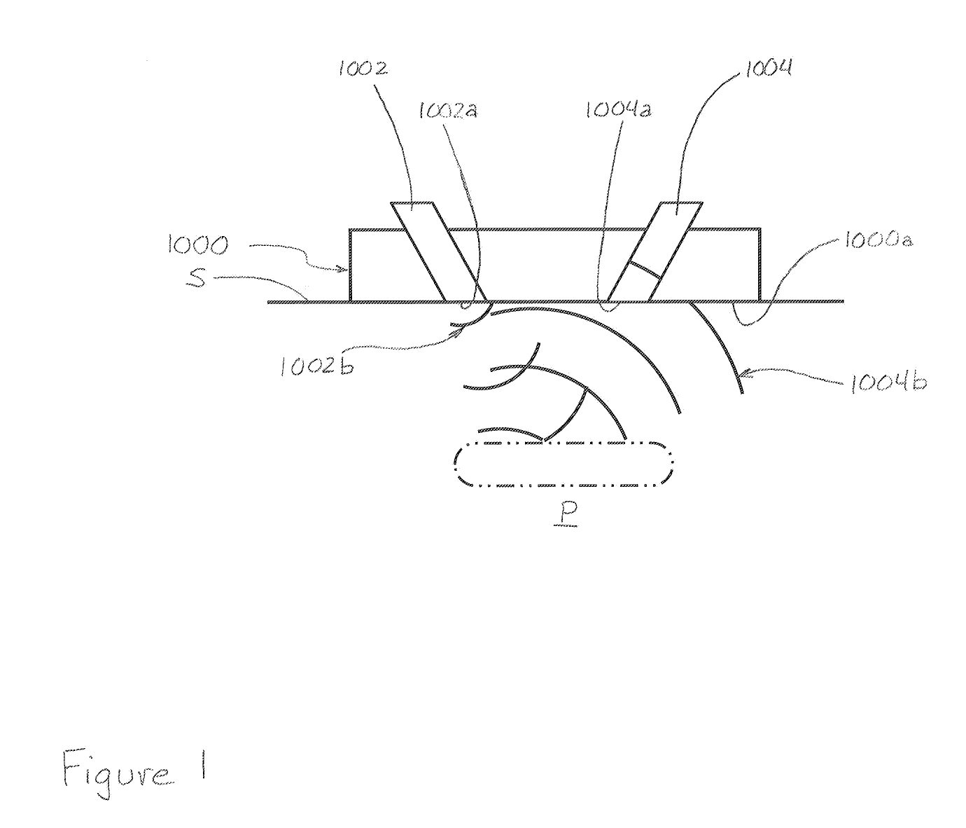

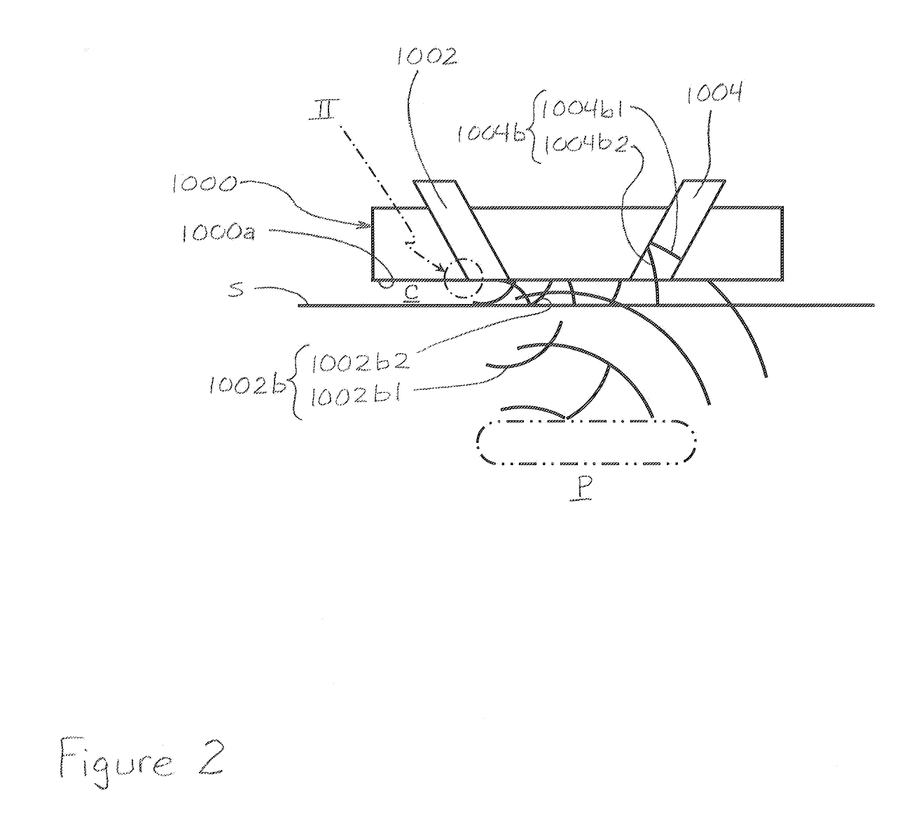

FIG. 1 shows an electromagnetic energy sensor 1000 preferably coupled with the skin S. According to one embodiment, electromagnetic energy sensor 1000 preferably operates in portions of the electromagnetic spectrum that include wavelengths generally not harmful to tissue, e.g., wavelengths longer than at least approximately 400 nanometers. Preferably, electromagnetic energy sensor 1000 operates in the visible radiation (light) or infrared radiation portions of the electromagnetic spectrum. According to other embodiments, electromagnetic energy sensor 1000 may operate in shorter wavelength portions of the electromagnetic spectrum, e.g., ultraviolet light, X-ray or gamma ray portions of the electromagnetic spectrum, preferably when radiation intensity and/or radiation duration are such that tissue harm is minimized.

Preferably, electromagnetic energy sensor 1000 includes an anatomic sensor. As the terminology is used herein, “anatomic” preferably refers to the structure of an Animalia body and an “anatomic sensor” preferably is concerned with sensing a change over time of the structure of the Animalia body. By comparison, a physiological sensor is concerned with sensing the functions and activities of an Animalia body, e.g., pulse, at a point in time.

Electromagnetic energy sensor 1000 preferably is arranged to overlie a target area of the skin S. As the terminology is used herein, “target area” preferably refers to a portion of a patient's skin that is generally proximal to where an infusate is being administered and frequently proximal to the cannulation site N. Preferably, the target area overlies the perivascular tissue P.

Electromagnetic energy sensor 1000 preferably uses electromagnetic radiation to aid in diagnosing infiltration or extravasation. Preferably, electromagnetic energy sensor 1000 includes an electromagnetic radiation signal transmitter 1002 and an electromagnetic radiation signal receiver 1004. Electromagnetic radiation signal transmitter 1002 preferably includes an emitter face 1002a for emitting electromagnetic radiation 1002b and electromagnetic radiation signal receiver 1004 preferably includes a detector face 1004a for detecting electromagnetic radiation 1004b. According to one embodiment, electromagnetic radiation signal transmitter 1002 preferably includes a set of first optical fibers and electromagnetic radiation signal receiver 1004 preferably includes a set of second optical fibers. Individual optical fibers in the first or second sets preferably each have end faces that form the emitter or detector faces, respectively. Preferably, emitted electromagnetic radiation 1002b from emitter face 1002a passes through the target area of the skin S toward the perivascular tissue P. Detected electromagnetic radiation 1004b preferably includes at least a portion of emitted electromagnetic radiation 1002b that is at least one of specularly reflected, diffusely reflected (e.g., due to scattering), fluoresced (e.g., due to endogenous or exogenous factors), or otherwise redirected from the perivascular tissue P before passing through the target area of the skin S to detector face 1004a. Preferably, an accumulation of fluid in the perivascular tissue P affects the absorption and/or scattering of emitted electromagnetic radiation 1002b and accordingly affects detected electromagnetic radiation 1004b. Accordingly, electromagnetic energy sensor 1000 preferably senses changes in detected electromagnetic radiation 1004b that correspond with anatomic changes over time, such as infiltration or extravasation of the perivascular tissue P.

Emitted and detected electromagnetic radiations 1002b and 1004b preferably are in the near-infrared portion of the electromagnetic spectrum. As the terminology is used herein, “near infrared” preferably refers to electromagnetic radiation having wavelengths between approximately 600 nanometers and approximately 2,100 nanometers. These wavelengths correspond to a frequency range of approximately 500 terahertz to approximately 145 terahertz. A desirable range in the near infrared portion of the electromagnetic spectrum preferably includes wavelengths between approximately 800 nanometers and approximately 1,050 nanometers. These wavelengths correspond to a frequency range of approximately 375 terahertz to approximately 285 terahertz. Emitted and detected electromagnetic radiations 1002b and 1004b preferably are tuned to a common peak wavelength. According to one embodiment, emitted and detected electromagnetic radiations 1002b and 1004b each have a peak centered about a single wavelength, e.g., approximately 970 nanometers (approximately 309 terahertz). According to other embodiments, emitted electromagnetic radiation 1002b includes a set of wavelengths in a band between a relatively short wavelength and a relatively long wavelength, and detected electromagnetic radiation 1004b encompasses at least the band between the relatively short and long wavelengths. According to still other embodiments, detected electromagnetic radiation 1004b is tuned to a set of wavelengths in a band between a relatively short wavelength and a relatively long wavelength, and emitted electromagnetic radiation 1002b encompasses at least the band between the relatively short and long wavelengths.

Electromagnetic energy sensor 1000 preferably includes a superficies 1000a that confronts the skin S. Preferably, superficies 1000a is generally smooth and includes emitter and detector faces 1002a and 1004a. As the terminology is used herein, “smooth” preferably refers to being substantially free from perceptible projections or indentations.

Electromagnetic energy sensor 1000 preferably is positioned in close proximity to the skin S. As the terminology is used herein, “close proximity” of electromagnetic energy sensor 1000 with respect to the skin S preferably refers to a relative arrangement that minimizes gaps between superficies 1000a and the epidermis of the skin S. Preferably, electromagnetic energy sensor 1000 contiguously engages the skin S as shown in FIG. 1.

The inventors discovered a problem regarding accurately identifying the occurrence of infiltration or extravasation because of a relatively low signal-to-noise ratio of detected electromagnetic radiation 1004b. In particular, the inventors discovered a problem regarding a relatively large amount of noise in detected electromagnetic radiation 1004b that obscures signals indicative of infiltration/extravasation events. Another discovery by the inventors is that the amount of noise in detected electromagnetic radiation 1004b tends to correspond with the degree of patient activity. In particular, the inventors discovered that detected electromagnetic radiation 1004b tends to have a relatively lower signal-to-noise ratio among patients that are more active, e.g., restless, fidgety, etc., and that detected electromagnetic radiation 1004b tends to have a relatively higher signal-to-noise ratio among patients that were less active, e.g., calm, sleeping, etc.

The inventors also discovered that a source of the problem is an imperfect cavity that may unavoidably and/or intermittently occur between superficies 1000a and the skin S. As the terminology is used herein, “imperfect cavity” preferably refers to a generally confined space that at least partially reflects electromagnetic radiation. In particular, the inventors discovered that the source of the problem is the imperfect cavity reflects portions of emitted electromagnetic radiation 1002b and/or detected electromagnetic radiation 1004b that are detected by electromagnetic radiation signal receiver 1004. Accordingly, detected electromagnetic radiation 1004b includes reflected extracorporeal electromagnetic radiation in addition to transcutaneous electromagnetic radiation. As the terminology is used herein, “extracorporeal electromagnetic radiation” generally refers to portions of emitted electromagnetic radiation 1002b and/or detected electromagnetic radiation 1004b that are reflected in the imperfect cavity, and “transcutaneous electromagnetic radiation” preferably refers to portions of emitted electromagnetic radiation 1002b that penetrate through the skin S and are reflected, scattered or otherwise redirected from the perivascular tissue P. Preferably, transcutaneous electromagnetic radiation includes a signal that indicates an infiltration/extravasation event whereas extracorporeal electromagnetic radiation predominately includes noise that tends to obscure the signal. Thus, the inventors discovered, inter alia, that a cavity between superficies 1000a and the skin S affects the signal-to-noise ratio of detected electromagnetic radiation 1004b.

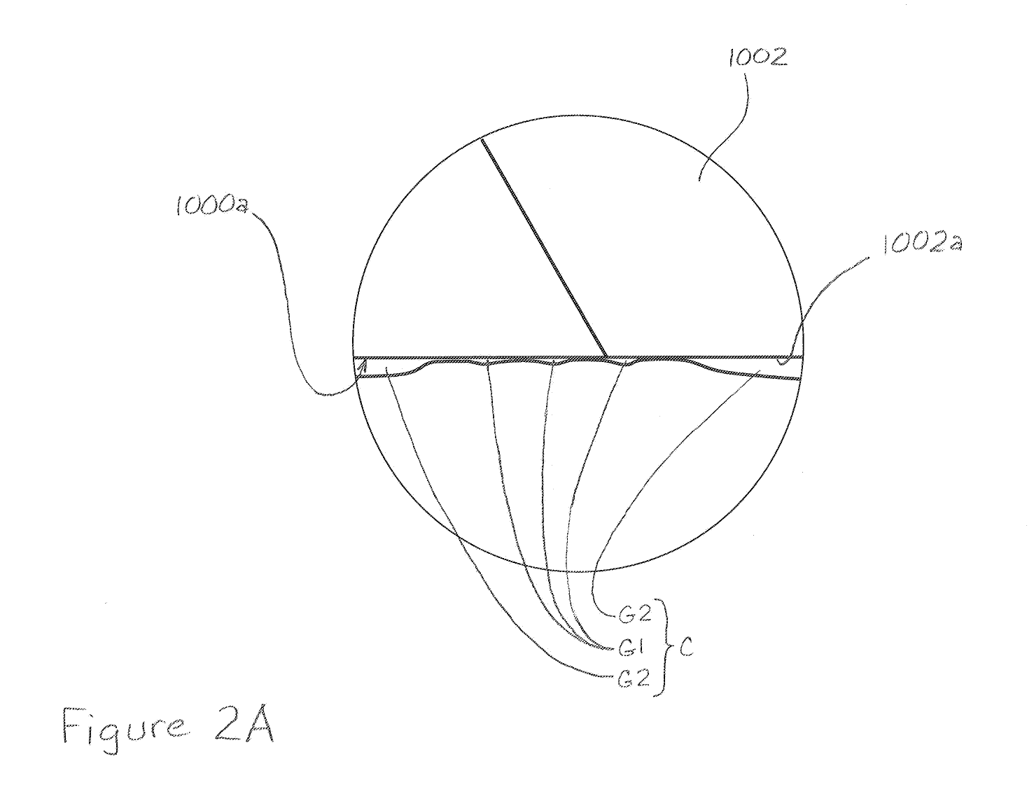

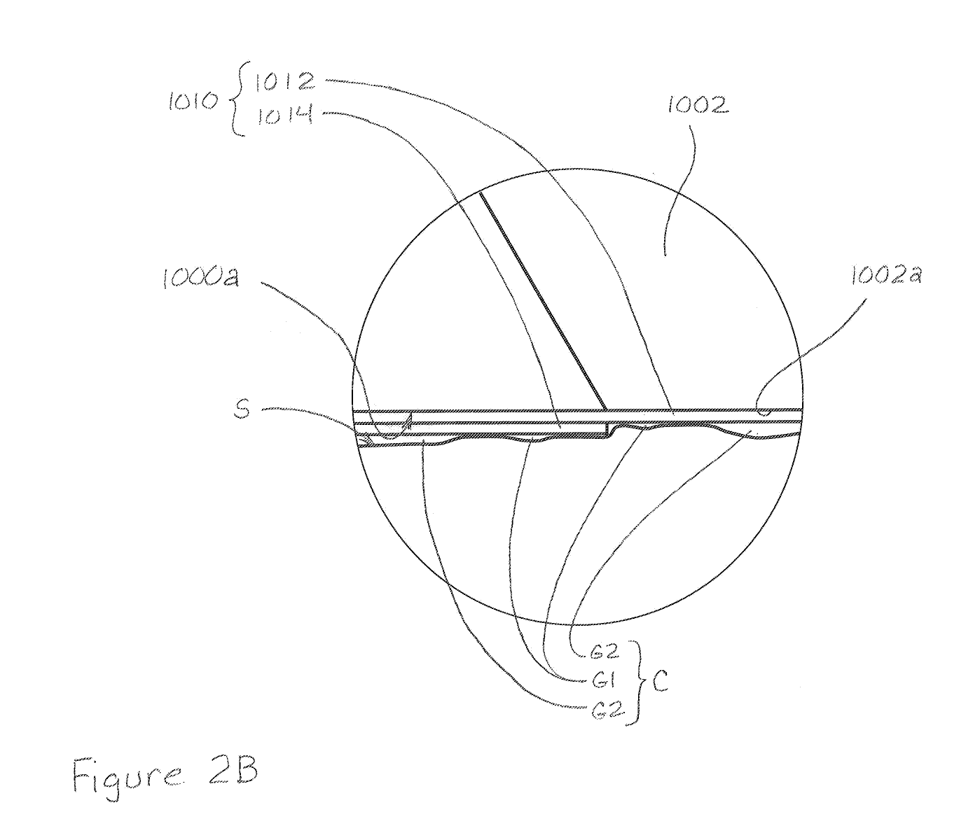

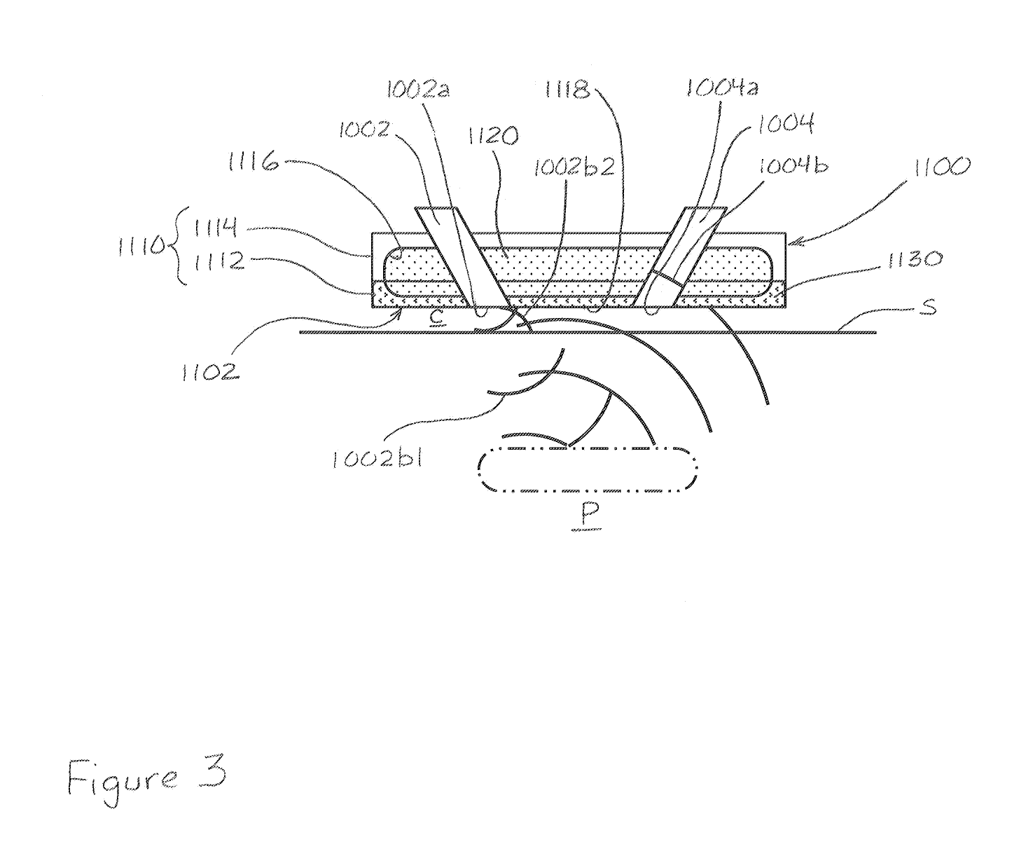

FIG. 2 illustrates the source of the problem discovered by the inventors. Specifically, FIG. 2 shows a cavity C disposed between electromagnetic energy sensor 1000 and the skin S. The size, shape, proportions, etc. of cavity C are generally overemphasized in FIG. 2 to facilitate describing the source of the problem discovered by the inventors. Preferably, emitted electromagnetic radiation 1002b includes a transcutaneous portion 1002b1 that passes through the cavity C and passes through the target area of the skin S toward the perivascular tissue P. Emitted electromagnetic radiation 1002b also includes an extracorporeal portion 1002b2 that is reflected in the cavity C. Detected electromagnetic radiation 1004b preferably includes signal 1004b1 as well as noise 1004b2. Preferably, signal 1004b1 includes at least a portion of transcutaneous portion 1002b1 that is at least one of reflected, scattered or otherwise redirected from the perivascular tissue P before passing through the target area of the skin S, passing through the cavity C, and being received by electromagnetic radiation signal receiver 1004. Noise 1004b2 includes at least a portion of extracorporeal portion 1002b2 that is reflected in the cavity C before being received by electromagnetic radiation signal receiver 1004.

FIGS. 2A and 2B illustrate that the cavity C preferably includes one or an aggregation of individual gaps. FIG. 2A shows individual gaps between superficies 1000a and the skin S that, taken in the aggregate, preferably make up the cavity C. Preferably, the individual gaps may range in size between approximately microscopic gaps G1 (three are indicated in FIG. 2A) and approximately macroscopic gaps G2 (two are indicated in FIG. 2A). It is believed that approximately microscopic gaps G1 may be due at least in part to epidermal contours of the skin S and/or hair on the skin S, and approximately macroscopic gaps G2 may be due at least in part to relative movement between superficies 1000a and the skin S. Patient activity is an example of an occurrence that may cause the relative movement that results in approximately macroscopic gaps G2 between superficies 1000a and the skin S.

FIG. 2B shows electromagnetic energy sensor 1000 preferably isolated from the skin S by a foundation 1010. Preferably, foundation 1010 contiguously engages superficies 1000a and contiguously engages the skin S. Accordingly, the cavity C between foundation 1010 and the skin S preferably includes an aggregation of (1) approximately microscopic gaps G1 (two are indicated in FIG. 2A); and (2) approximately macroscopic gaps G2 (two are indicated in FIG. 2A). Foundation 1010 preferably is coupled with respect to electromagnetic energy sensor 1000 and includes a panel 1012 and/or adhesive 1014. Preferably, panel 1012 includes a layer disposed between electromagnetic energy sensor 1000 and the skin S. Panel 1012 preferably includes Tegaderm™, manufactured by 3M (St. Paul, Minn., USA), REACTIC™, manufactured by Smith & Nephew (London, UK), or another polymer film, e.g., polyurethane film, that is substantially impervious to solids, liquids, microorganisms and/or viruses. Preferably, panel 1012 is transparent or translucent with respect to visible light, breathable, and/or biocompatible. As the terminology is used herein, “biocompatible” preferably refers to compliance with Standard 10993 promulgated by the International Organization for Standardization (ISO 10993) and/or Class VI promulgated by The United States Pharmacopeial Convention (USP Class VI). Other regulatory entities, e.g., National Institute of Standards and Technology, may also promulgate standards that may additionally or alternatively be applicable regarding biocompatibility. Panel 1012 preferably is generally transparent with respect to emitted and detected electromagnetic radiations 1002b and 1004b. Preferably, adhesive 1014 bonds at least one of panel 1012 and electromagnetic energy sensor 1000 to the skin S. Adhesive 1014 preferably includes an acrylic adhesive, a synthetic rubber adhesive, or another biocompatible, medical grade adhesive. Preferably, adhesive 1014 minimally affects emitted and detected electromagnetic radiations 1002b and 1004b. According to one embodiment, as shown in FIG. 2B, adhesive 1014 preferably is omitted where emitted and detected electromagnetic radiations 1002b and 1004b penetrate foundation 1010, e.g., underlying emitter and detector faces 1002a and 1004a.

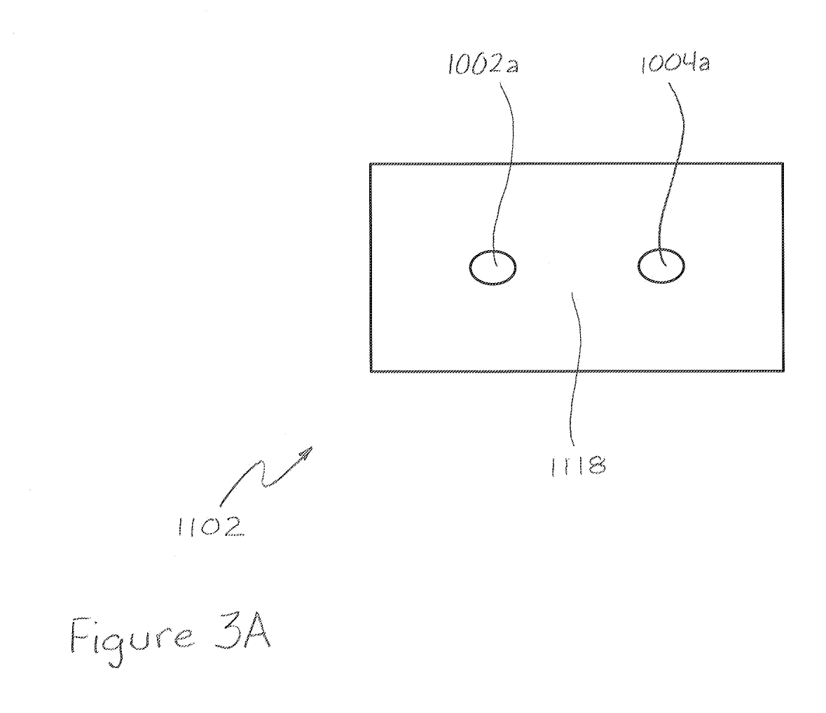

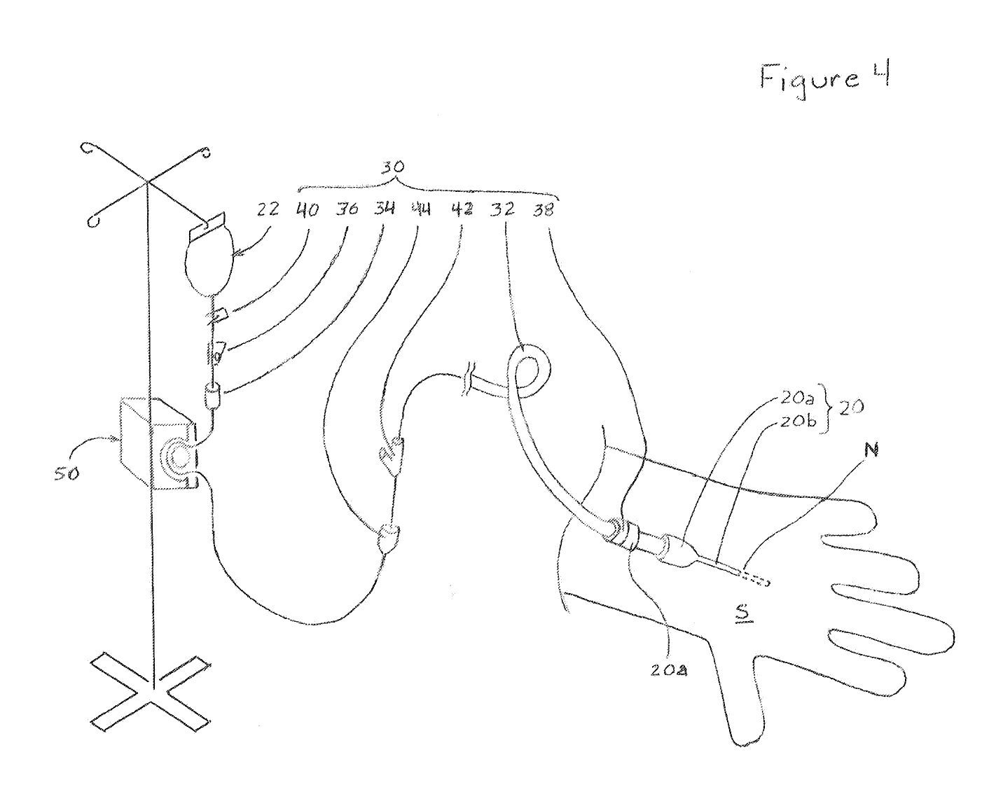

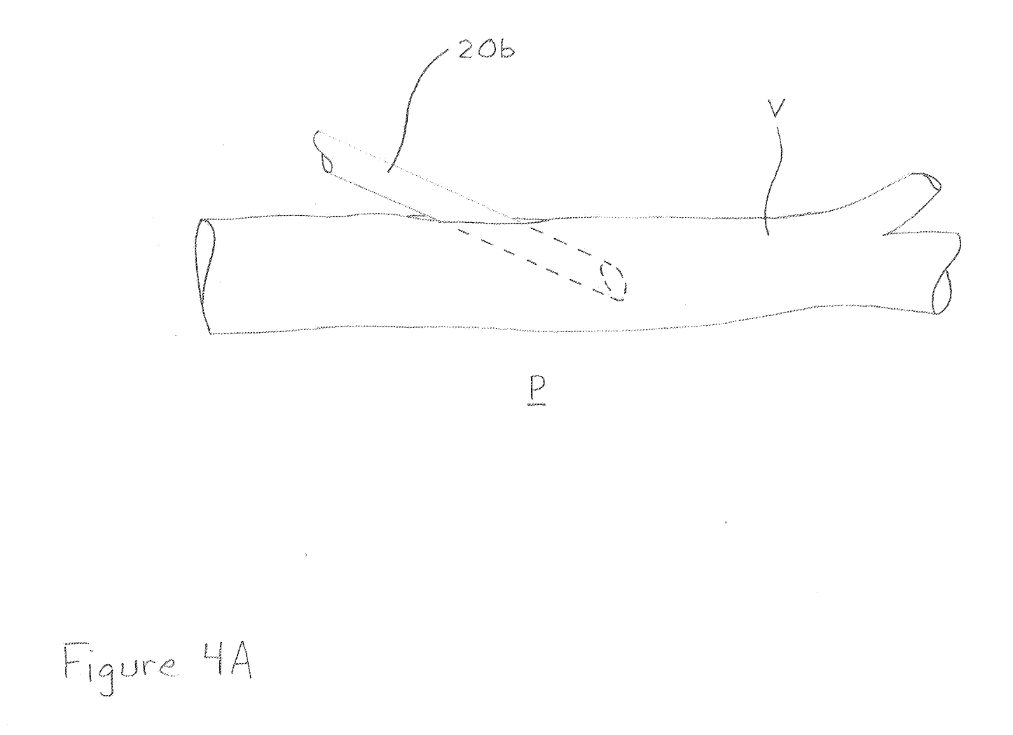

FIG. 3 shows an electromagnetic energy sensor 1100 according to the present disclosure that preferably includes a housing 1110 with an electromagnetic radiation absorber 1130. According to one embodiment, housing 1110 preferably includes a first housing portion 1112 coupled with a second housing portion 1114. Preferably, electromagnetic radiation signal transmitter 1002 and electromagnetic radiation signal receiver 1004 extend through a space 1116 generally defined by housing 1110. Housing 1110 preferably includes a biocompatible material, e.g., polycarbonate, polypropylene, polyethylene, acrylonitrile butadiene styrene, or another polymer material. A potting material 1120, e.g., epoxy, preferably fills space 1116 around electromagnetic radiation signal transmitter 1002 and electromagnetic radiation signal receiver 1004. According to one embodiment, potting material 1120 preferably cinctures transmitting and receiving optical fibers disposed in space 1116. Preferably, housing 1110 includes a surface 1118 that confronts the skin S and cinctures emitter and detector faces 1002a and 1004a. Accordingly, as shown in FIG. 3A, a superficies 1102 of electromagnetic energy sensor 1100 preferably includes emitter face 1002a, detector face 1004a and surface 1118.

Absorber 1130 preferably absorbs electromagnetic radiation that impinges on surface 1118. As the terminology is used herein, “absorb” or “absorption” preferably refer to transforming electromagnetic radiation to another form of energy, such as heat, while propagating in a material. Preferably, absorber 1130 absorbs wavelengths of electromagnetic radiation that generally correspond to the wavelengths of emitted and detected electromagnetic radiations 1002b and 1004b. According to one embodiment, absorber 1130 preferably absorbs electromagnetic radiation in the near-infrared portion of the electromagnetic spectrum. Absorber 1130 may additionally or alternatively absorb wavelengths in other parts of the electromagnetic radiation spectrum, e.g., visible light, short-wavelength infrared, mid-wavelength infrared, long-wavelength infrared, or far infrared. Preferably, absorber 1130 absorbs at least 50% to 90% or more of the electromagnetic radiation that impinges on surface 1118.

Absorber 1130 preferably includes a variety of form factors for inclusion with housing 1110. Preferably, absorber 1130 includes at least one of a film, a powder, a pigment, a dye, or ink. Film or ink preferably are applied on surface 1118, and powder, pigment or dye preferably are incorporated, e.g., dispersed, in the composition of housing 1110. FIG. 3 shows absorber 1130 preferably is included in first housing portion 1112; however, absorber 1130 or another electromagnetic radiation absorbing material may also be included in second housing portion 1114 and/or potting material 1120. Examples of absorbers 1130 that are suitable for absorbing near-infrared electromagnetic radiation preferably include at least one of antimony-tin oxide, carbon black, copper phosphate, copper pyrophosphate, illite, indium-tin oxide, kaolin, lanthanum hexaboride, montmorillonite, nickel dithiolene dye, palladium dithiolene dye, platinum dithiolene dye, tungsten oxide, and tungsten trioxide.

Absorber 1130 preferably improves the signal-to-noise ratio of received electromagnetic radiation 1004 by reducing noise 1004b2. Compared to electromagnetic energy sensor 1000 (FIG. 2), the propagation of extracorporeal portion 1002b2 preferably is substantially attenuated by absorber 1130 in electromagnetic energy sensor 1100. Preferably, extracorporeal portion 1002b2 that impinges on surface 1118 is absorbed rather than being reflected in the cavity C and therefore does not propagate further, e.g., toward electromagnetic radiation signal receiver 1004. Other electromagnetic radiation that impinges on surface 1118 preferably is also absorbed rather than being reflected in the cavity C. For example, absorber 130 may also absorb a portion of transcutaneous portion 1002b1 that is at least one of reflected, scattered or otherwise redirected from the perivascular tissue P, then passes through the target area of the skin S and through the cavity C, but impinges on surface 1118 rather than being received by electromagnetic radiation signal receiver 1004.

Electromagnetic energy sensor 1100 preferably may be used, for example, (1) as an aid in detecting at least one of infiltration and extravasation; (2) to identify an anatomical change in perivascular tissue; or (3) to analyze a transcutaneous electromagnetic signal. Preferably, electromagnetic radiation signal transmitter 1002 transmits emitted electromagnetic radiation 1002b via emitter face 1002a. Emitted electromagnetic radiation 1002b preferably propagates through foundation 1010 and/or cavity C, if either of these is disposed in the path of emitted electromagnetic radiation 1002b toward the target area of the skin S. According to one embodiment, emitted electromagnetic radiation 1002b divides into transcutaneous portion 1002b1 and extracorporeal portion 1002b2 in the cavity C.

Transcutaneous portion 1002b1 of emitted electromagnetic radiation 1002b preferably propagates through the skin S toward the perivascular tissue P. Preferably, at least a portion of transcutaneous portion 1002b1 is at least one of reflected, scattered or otherwise redirected from the perivascular tissue P toward the target area of the skin S as signal 1004b1. After propagating through the target area of the skin S, signal 1004b1 preferably further propagates through the cavity C and foundation 1010, if either of these is disposed in the path of signal 1004b1 toward electromagnetic radiation signal receiver 1004. Preferably, electromagnetic radiation signal receiver 1004 receives signal 1004b1 via detector face 1004a. Signal 1004b1 preferably includes a transcutaneous electromagnetic signal that may be analyzed to, for example, identify anatomical changes in perivascular tissue and/or aid in detecting an infiltration/extravasation event.

Extracorporeal portion 1002b2 of emitted electromagnetic radiation 1002b is reflected in cavity C, but preferably is generally absorbed by absorber 1130. Preferably, absorber 1130 absorbs at least 50% to 90% or more of extracorporeal portion 1002b2 that impinges on surface 1118. Accordingly, a first portion of noise 1004b2 due to extracorporeal portion 1002b2 preferably is substantially eliminated or at least reduced by absorber 1130.

Absorber 1130 preferably also absorbs a second portion of noise 1004b2 due to electromagnetic radiation other than extracorporeal portion 1002b2 in cavity C. For example, absorber 1130 preferably also absorbs a portion of signal 1004b1 that impinges on surface 1118 rather than being received by electromagnetic radiation signal receiver 1004 via detector face 1004a.

Thus, absorber 1130 preferably improves the signal-to-noise ratio of detected electromagnetic radiation 1004b by absorbing noise 1004b2. Preferably, reducing noise 1004b2 in detected electromagnetic radiation 1004b makes it easier to analyze signal 1004b1 in detected electromagnetic radiation 1004b.

Changes in the size and/or volume of cavity C preferably may also be used to monitor patient activity and/or verify inspections by caregivers. Preferably, information regarding the frequency and degree of patient motion may be detected by electromagnetic energy sensor 1100. Accordingly, this information may aid a caregiver in evaluating if a patient is obsessed with or distracted by cannula 20 and therefore at greater risk of disrupting the patient's infusion therapy. Similarly, electromagnetic energy sensor 1100 preferably may be used to detect caregiver inspections of the target area of the skin and/or the insertion site N. Preferably, a caregiver periodically inspects the patient during infusion therapy for indications of infiltration/extravasation events. These inspections preferably include touching and/or palpitating the target area of the patient's skin; which tends to cause relative movement between electromagnetic energy sensor 1100 and the skin. Accordingly, a record of detected electromagnetic radiation 1004b preferably includes the occurrences over time of caregiver inspections.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A sensor for evaluating an anatomical change over time in perivascular tissue, the sensor comprising:
an emitter face configured to emit a first electromagnetic radiation signal;
a detector face configured to detect a second electromagnetic radiation signal, the second electromagnetic radiation signal being at least one of a reflection, scattering and redirection of the first electromagnetic radiation signal by the perivascular tissue; and
an absorber configured to absorb a third electromagnetic radiation signal, the third electromagnetic radiation signal being at least one of a reflection, scattering and redirection of the first electromagnetic radiation signal by epidermis overlying the perivascular tissue.

2. The sensor of claim 1, comprising a superficies configured to confront the epidermis, the superficies includes the emitter face, the detector face, and a surface of the absorber.

3. The sensor of claim 2 wherein the surface cinctures the emitter and detector faces.

4. The sensor of claim 1, comprising a foundation disposed between the superficies and the epidermis.

5. The sensor of claim 4 wherein the foundation is configured to be substantially transparent to the first and second electromagnetic radiation signals.

6. The sensor of claim 4 wherein the foundation is configured to be substantially transparent to the first, second and third electromagnetic radiation signals.

7. The sensor of claim 1 wherein the first, second and third electromagnetic radiation signals comprise near-infrared signals.

8. The sensor of claim 7 wherein the absorber comprises a near-infrared absorber configured to absorb a band of electromagnetic radiation signals having wavelengths between approximately 600 nanometers and approximately 2,100 nanometers.

9. The sensor of claim 7 wherein the absorber comprises a near-infrared absorber configured to absorb a band of electromagnetic radiation signals having wavelengths between approximately 600 nanometers and approximately 1,800 nanometers.

10. The sensor of claim 9 wherein the band of electromagnetic radiation signals have wavelengths between approximately 800 nanometers and approximately 1,050 nanometers.

11. The sensor of claim 1, comprising pluralities of emitter and detector optical fibers, the emitter face includes individual faces of the plurality of emitter optical fibers, and the detector end face includes individual faces of the plurality of detector optical fibers.

12. The sensor of claim 11, comprising first and second housing portions, the first housing portion includes the absorber, and the second housing portion is coupled to the first housing portion.

13. The sensor of claim 12 wherein at least one of the first and second housing portions comprises a polymer and the absorber is generally dispersed in the polymer.

14. The sensor of claim 13 wherein the polymer consists of at least one of polycarbonate, polypropylene, polyethylene and acrylonitrile butadiene styrene.

15. The sensor of claim 12 wherein the first and second housing portions generally define a space, and the pluralities of emitter and detector optical fibers extend through the space.

16. The sensor of claim 15, comprising a potting material being disposed in the space and cincturing the pluralities of emitter and detector optical fibers.

17. The sensor of claim 1 wherein the absorber comprises at least one of a film, a powder, a pigment, a dye, and ink.

18. The sensor of claim 1 wherein the absorber comprises at least one of antimony-tin oxide, carbon black, copper phosphate, copper pyrophosphate, illite, indium-tin oxide, kaolin, lanthanum hexaboride, montmorillonite, nickel dithiolene dye, palladium dithiolene dye, platinum dithiolene dye, tungsten oxide, and tungsten trioxide.

19. The sensor of claim 1 wherein the second electromagnetic radiation signal is configured to aide in diagnosing at least one of infiltration and extravasation.

20. The sensor of claim 1 wherein absorbing the third electromagnetic radiation signal improves a signal-to-noise ratio of the second electromagnetic radiation signal.
